(19) United States
(12) Patent Application Publication
(10) Pub. No.: US 2009/0241966 A1
(43) Pub. Date: Oct. 1, 2009

(54) Condom Packaging for Singular Direction of Removal of Contents

(76) Inventors: Jordan Gray, Dublin, OH (US); Robert Smith, Fort Lauderdale, FL (US)

Correspondence Address: Allen F. Bennett;Santucci, Priore & Long, LLP, 200 South Andrews, SUITE 100, FORT LAUDERDALE, FL, 33301, US

(21) Appl. No.: 12/056,647

(22) Filed: Mar. 27, 2008

(51) Int. Cl. A61F 6/04; B65D 85/08

(52) U.S. Cl. 128/844; 206/69

(57) ABSTRACT

A condom package for a singular direction or removal of the contents thereof to assure its proper application onto a user. The package includes a container having a configuration with an essentially flat top side and a bottom side, the top side and the bottom side each having an external surface and an internal surface, the external surfaces of the top side and the bottom side facing in opposing directions, the internal surfaces facing each other, the internal surfaces having peripheral portions removably sealed to each other. The bottom side formed of a material more rigid than that of the top side, the bottom side having a shallow central well proportioned to hold and stabilize the condom.

### BACKGROUND

1. Field of the Invention

The present invention relates to condom packaging. More particularly, the invention relates to condom packaging with a singular method to access its contents to pre-determine the orientation of a condom to ensure proper application to the user of the condom.

2. Prior Art

At the present time, there is much emphasis and advertising concerning the “consistent and correct use” of condoms for health purposes, birth control, and the spread of disease, most notably HIV/AIDS. Typically, condoms are made of an elastic material, such as latex, polyurethane, or natural animal membranes, and are packaged individually in a sealed plastic or foil pouch. When packaged, the condom is rolled in a manner such that it appears as a latex disk with a tubular or torodial edge.

Use of the condom typically involves opening the packaging, removing the condom from the package, visually determining the correct orientation of the condom, and application of the condom. Condoms are orientation specific with regard to the direction in which they will unroll. However, if one tries to unroll the condom in the wrong direction, it is usually not at first visually apparent that one has done so. A visual determination is usually also impaired by low lighting conditions, or always impaired for users populations with significant visual deficits.

Once a user starts to incorrectly apply the condom, it becomes obvious to the user since the condom will not unroll. At this point it is not unlikely for a user to simply reverse the direction of the condom. Apart from the frustration and interruption, there is also a danger that semen and/or a pathogen may have been deposited on the outer surface when the user first tried to unroll the condom, and it may be thereafter transmitted to the user's sexual partner. This particular risk of contamination relates directly to the fact that most condom failures result from incorrect use of the condom, not because the condom itself was faulty.

It is known to package condoms in a manner which a user may distinguish, by touch using ridges or stickers, the orientation of the condom as shown in GB 2,219,782 to Ball. U.S. Pat. No. 5,666,972 to Gifford shows a condom package with tactile means of identifying the orientation of the condom within the package through ridges and clipped corners located on the outer packaging.

U.S. Pat. No. 5,044,492 to Auerbach illustrates a package for an individual condom with an exterior tactile indication of the orientation of the condom within. The indication is one or more protrusions formed in the material of the package or a piece of suitable material adhesively applied. It also exhibits a vertical cross-sectional symmetry which can contribute to confusion after the package is opened, and limits physical protection by the packaging of the condom itself.

WO 03/037232 to Gligoric shows a condom sack with one external side that has a rib-wrinkled surface, marked with symbols in the form of a circle with arrows, and normal/fluorescent colors. This particular reference again is aimed to assist the user in correctly applying the condom by using visual and tactile cues. It is therefore desirable to provide a simple means for determining the correct orientation of the condom by using one's sense of touch and of stabilizing the condom prior to use. It is also desirable to provide a simple means for determining the correct orientation of the condom by using one's sense of sight. The present invention improves upon those strategies for addressing this problem, known in the art.

### SUMMARY OF THE INVENTION

A condom package for a singular direction of removal of the contents thereof to assure an initial proper application onto a user and thereby, its “consistent and correct use”. The package includes a container having a configuration with a flat top side and a bottom side, the top side and the bottom side each having an external surface and an internal surface, the external surfaces of the top side and the bottom side facing in opposing directions, the internal surfaces facing each other, the internal surfaces having peripheral portions removably sealed to each other. The bottom side formed of a material more rigid than that of the top side, the bottom side having a shallow depression or central well proportioned to hold and stabilize the condom.

It is an object of the invention to provide a condom packaging with a method to access its contents that pre-determines the orientation of a condom prior to its use and this minimizes the possibility of misuse thereof. If the object of this invention is achieved, greater populations of individuals will be able to access the condom “consistent and correct use” even in low lighting, or if individuals have any partial, or significant loss of vision, tactile, stereognosis or fine motor control that would be required to open typical condom packaging and correctly orient the condom before use.

The above and yet other objects and advantages of the present invention will become apparent from the hereinafter set forth Brief Description of the Drawings, Detailed Description of the Invention and the Claims appended herewith.

### BRIEF DESCRIPTION OF THE DRAWINGS

FIG. 1 is a perspective assembly view of the condom packaging showing a peel-away stop side thereof.

FIG. 2 is an exploded view of the packaging of FIG. 1.

FIG. 3 is a vertical, cross-sectional, exploded view of a second embodiment of the invention.

FIG. 4 is a vertical cross-sectional exploded view of a third embodiment thereof.

FIG. 5 is a perspective assembly view of a fourth embodiment.

FIG. 6 is a perspective assembly view of a fifth embodiment of the invention.

### DETAILED DESCRIPTION OF THE INVENTION

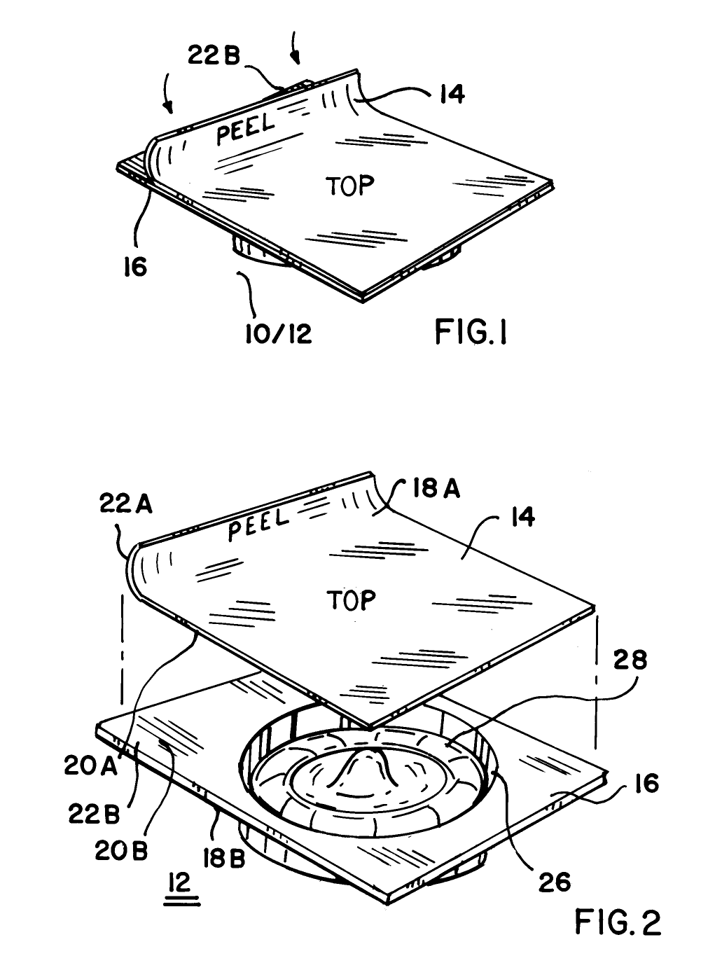

With reference to the perspective view of FIG. 1, there is shown a condom package 10 which sets forth a singular direction for access to the contents thereof, the purpose being to assure application in the proper direction of the condom onto a user thereof. The inventive package 10 more particularly comprises a container 12 including a configuration having a flat top side 14 and a particularly pre-formed bottom side 16, as is more particularly described below. Said top side 14 and bottom side 16 each exhibit external surfaces 18A and 18B respectively and internal surfaces 20A and 20B respectively which face in opposing directions, that as may be noted in FIGS. 2-4. Said opposing internal surfaces 20A and 20B include peripheral portions 22A and 22B which are removably sealed to each other at the time of manufacture, that is, are sealed at the time of manufacture but are removably peeled away from each other upon the occasion of use. The inventive condom package is particularly characterized in that said bottom side 16 is formed of a material which is more rigid than that of top side 14, this having various benefits as are fully noted below, one thereof being that a less rigid surface can be more readily peeled away from a more rigid surface when the two are press formed together with a contact adhesive, as is the case in present system. The same would equally be the case if top side 14 were adhered to bottom side 16 in a different fashion, for example, by a multiplicity of pressure points or perforations therebetween.

The instant invention is further characterized in that the inventive condom package 10 includes a shallow central well 26. As may be noted in FIG. 3, said shallow central well 26 is proportioned to hold and stabilize a condom 28 therewithin so that, after removal of top side 14 from bottom side 16, peripheral portion 22 of bottom side 16 may be engaged by the fingers of one hand of the user while condom 28 is removed by the other hand so that, in a single motion (if desired by the user), it can then be applied over the penis of the user without concern that its axial orientation may be incorrect. This of course is not the case in conventional condom packaging wherein it is often difficult, particularly under conditions of poor lighting, to ascertain the so-called top from the bottom of the condom, the result being potentially an unintended pregnancy, or cross infection consequences, as are more fully set forth in the Background of the Invention above. It may, from the views of FIGS. 1-3 be seen that condom container 12 includes top and bottom sides which are preferably square and substantially congruent with each other. Such of course is not essential and, for example, as shown in FIG. 3, the horizontal cross-sectional dimension of upper or top side 14 may be slightly less than that of lower or bottom side 16 in order to make it easier for a user to engage the peripheral edge of the more rigid lower surface of bottom side 16 and to peel-up portion 22A of top side 14.

In terms of packaging of the inventive condom package 10 within a larger carton for retail sale, a plurality of several of the inventive condom packages as set forth herein may be connected to each other by perforations such that individual unit packages may be separated by the user at a time convenient to him.

In terms of materials, top side 14 may be formed of a thick aluminum foil, thereby permitting easing of tearing, product hygiene and product integrity relative to the above-described bottom side 16 which, for example, can be formed of a rigid but slightly flexible plastic such as a PVC polymer.

In FIG. 3 is further shown tab 22 which may be held by the fingers of one hand of a user while top side 14 is peeled-off. Holding tab 22 also helps to assure proper orientation of condom 28 when it is removed from well 26.

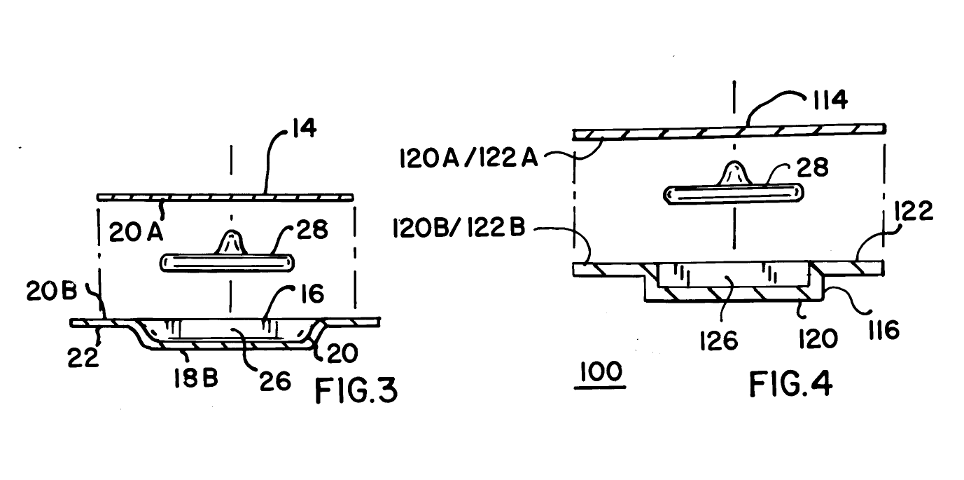

In FIG. 4 is shown an alternative embodiment of the invention in which shallow central volume 26 of FIGS. 1-3 is replaced by a solid rectangular volume 126. This approach may provide for greater stability of condom 28 within the bottom or lower side 116 of the condom package 100 after the top of the condom package is peeled away. Further, the embodiment of FIG. 4 lends itself to use with a thicker and/or more rigid polymeric material to thereby further protect condom 28 from external forces and pressure and to provide a more fluid integral enclosure so that the condom may remain more suitably during what may be an extended shelf life of the product prior to use. Also provided in this embodiment are tabs 122B which may be held by the fingers of one hand of the user while the condom is removed.

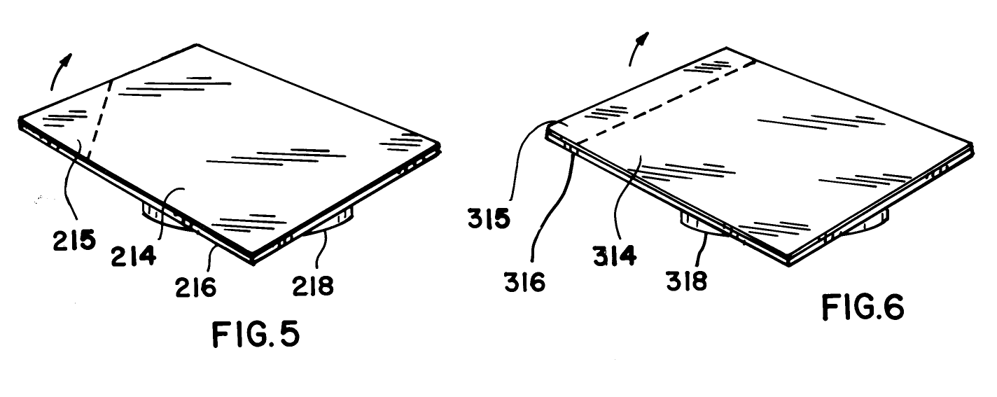

As may be noted in the view of FIGS. 5 and 6, various forms of breakaway removal of top side relative to bottom side may be employed. More particularly, FIG. 5 demonstrates a strategy in which a corner 215 of top side 214 may be readily peeled-off of bottom side 216. This, to some users, may be more convenient that the classical peel strategy shown in FIGS. 1 and 2 given that it is always possible that the adhesive connecting the top and the bottom side may become too strong, adhered or the like thereby rendering it difficult to separate the top from the bottom side of the package. Whereas, in a breakaway strategy, this problem is less likely to occur. An alternative breakaway opening package is shown in FIG. 6, this being more directly analogous to the peel opening strategy along an entire edge of the package which is shown in FIGS. 1 and 2.

While there has been shown and described the preferred embodiment of the instant invention it is to be appreciated that the invention may be embodied otherwise than is herein specifically shown and described and that, within said embodiment, certain changes may be made in the form and arrangement of the parts without departing from the underlying ideas or principles of this invention as set forth in the Claims appended herewith.

We claim:

1. A condom package having a singular directions of removal of the contents thereof to assure its proper application onto a user, the package comprising:
a container having a flat top side and a bottom side, said top side and said bottom side each having an external surface and an internal surface, said external surfaces of said top side and said bottom side facing in opposing directions, said internal surfaces facing each other, said internal surfaces having peripheral portions removably sealed to each other, said bottom side comprising a material more rigid than that of said top side, said bottom side having a shallow central well proportioned to hold and stabilize a condom therein.

2. A condom as recited in claim 1, in which said top and bottom sides have a square, substantially congruent configuration.

3. A condom as recited in claim 2, in which said square configuration comprises a side having perforations to connect two or more of said packages to each other.

4. A condom package as recited in claim 1, in which said top side comprises an aluminum foil or other material exhibiting ease of tearing, product hygiene and product integrity.

5. A condom package as recited in claim 1, in which said bottom side is made of a substantially rigid polymeric material.

6. A condom package as recited in claim 1, in which said top side includes a potentially smaller area relative to said bottom side.

7. A condom package as recited in claim 1, in which at least one portion of said top side comprises means to remove said top side.

8. A condom package as recited in claim 1, in which at least one surface of said bottom side comprises a peripheral tab.

9. A condom package as recited in claim 6, in which at least one surface of said bottom side comprises a peripheral tab.

10. A condom package as recited in claim 1, in which a peripheral extent of said top side is lesser than a peripheral extent of said bottom side
